SEQUENCE LISTING (10 sequences)

SEQ ID NO: 1 - DNA, 776 bp, Lactobacillus rhamnosus
tatacactggtacctccctaagtgggatacattgaaacaatctatccgcataatcaagaccgcatgtcttgctaagatgcgtaactatcgctttggatgaccccgcgtatagctagttgtaagtaacgctcaccaagcaatgatgctagccaactaagttgatcgccacattggactaaacacggcccaaactctacggaggcagcagtaggaatcttccacaatggacgcaagtctgatggagcaacgccgcgtgactgaagaaggctttcggggcgtaaaactctgttgttggagaagaatggtcggcagagtaactgttgtcggcgtgacggtatccaaccagaaagccacggctaactcagtgccagcagccgcggtaatacgtaggtggcaagcgttatccggatttattgggcgtaaagcgagcgcaggcggttttttaactctgatgtgaaagccctcggcttaaccgaggaagtgcatcggaaactgggaaacttgagtacagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcactaacgcattaagcattccgcctggggagtacgaccgcaaggttgaaactcaaaggaattgacgg

SEQ ID NO: 2 - DNA, 581 bp, Lactobacillus rhamnosus
ttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctttctaaggaaacagactgaaagtctgacggaaacctgcacacacgaaactttgtttagttttgaggggattaccctcaagcaccctagcgggtgcgactttgttctttgaaaactggatatcattgttgtaaatgttttaaattgccgagaacacagcgtatttgtatgagtttctaataatagaaattcgcatcgcataaccgctgacgcaagtcagtaccaggtaagttacaaagggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctaag

SEQ ID NO: 3 - DNA, 589 bp, Lactobacillus rhamnosus
cttgtacacaccgcccgtcacaccatgagagtttgtaacacccgaagccggtggcgtaaccttttagggagcgagccgtctaaggtgggacaaatgattagggtgaagtcgtaacaaggtagccgtaggagaacctgcggctggatcacctcctttctaaggaaacagactgaaagtctgacggaaacctgcacacacgaaactttgtttagttttgaggggatcaccctcaagcaccctaacgggtgcgactttgttctttgaaaactggatatcattgtattaattgttttaaattgccgagaacacagcgtatttgtatgagtttctgaaaaagaaattcgcatcgcataaccgctgacgcaagtcagtacaggttaagttacaaagggcgcacggtggatgccttggcactaggagccgatgaaggacggaactaataccgatatgcttcggggagctataagtaagctttgatccggagatttccgaatgggggaacccagtacacatcagtgtgttgcttgtcagtgaatacatagctggccggcggccagacgcggggaactgaaacatctaagtacccgga

SEQ ID NO: 4 - DNA, 686 bp, Lactobacillus rhamnosus
cctttctaaggaaacagactgaaagtctgacggaaacctgcacacacgaaactttgtttagttttgaggggattaccctcaagcaccctagcgggtgcgactttgttctttgaaaactggatatcattgttgtaaatgttttaaattgccgagaacacaggctatttgtatgagtttctaataatagaaattcgcatcgcataaccgctgacgcaagtcagtacaggttaagttacaaagggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagttcgacgtagtcgaagtcagctggaaagctgcgccatagaaggtgaaagccctgtaaacgaaacggcggactctccgtccaggatcctgagtacggcggaacacgtgaaattccgtcggaatccgggaggaccatct

SEQ ID NO: 5 - DNA, 222 bp, Lactobacillus rhamnosus
ctaaggaaacagactgaaagtctgacggaaacctgcacacacgaaactttgtttagttttgaggggattaccctcaagcaccctagcgggtgcgactttgttctttgaaaactggatatcattgttgtaaatgttttaaattgccgagaacacagcgtatttgtatgagtttctaataatagaaattcgcatcgcataaccgctgacgcaagtcagtacagg

SEQ ID NO: 6 - DNA, 222 bp, Lactobacillus rhamnosus; identical to SEQ ID NO: 5

SEQ ID NO: 7 - DNA, 218 bp, Lactobacillus rhamnosus
aaggaaacagactgaaagtctgacggaaacctgcacacacgaaactttgtttagttttgaggggattaccctcaagcaccctagcgggtgcgactttgttctttgaaaactggatatcattgttgtaaatgttttaaattgccgagaacacagcgtatttgtatgagtttctaataatagaaattcgcatcgcataaccgctgacgcaagtcagtaca

SEQ ID NO: 8 - DNA, 24 bp, Artificial Sequence
  Description of Artificial Sequence Primer
cccactgctgcctcccgtaggagt

SEQ ID NO: 9 - DNA, 20 bp, Artificial Sequence
  Description of Artificial Sequence Primer
tgcatcttgatttaattttg

SEQ ID NO: 10 - DNA, 20 bp, Artificial Sequence
  Description of Artificial Sequence Primer
ccgtcaattcctttgagttt

(19) United States
(12) Patent Application Publication
(10) Pub. No.: US 2005/0074440 A1
(43) Pub. Date: Apr. 7, 2005

(54) Lactobacillus rhamnosus strain and uses thereof

(75) Inventor: Chih-Feng Lin, Taipei City (TW)

Correspondence Address: FOLEY AND LARDNER;SUITE 500, 3000 K STREET NW, WASHINGTON, DC, 20007, US

(73) Assignee: T-CELL BIOTECHNOLOGY FOOD CO., LTD.

(21) Appl. No.: 10/624,495

(22) Filed: Jul. 23, 2003

Related U.S. Application Data
(63) Continuation of application No. 09/686,067, filed on Oct. 10, 2000, now abandoned.

(30) Foreign Application Priority Data
Jul. 29, 2000 (TW) 089115288

(51) Int. Cl. A61K 45/00; A61K 47/00; C12N 1/20

(52) U.S. Cl. 424/93.45; 424/439; 435/252.9

(57) ABSTRACT

A novel strain of Lactobacillus rhamnosus is disclosed, which is phylogenetically distinct from the published strains in the species and exhibits excellent probiotic properties. The medical and nutritional uses of the bacterial strain are also disclosed.

### BACKGROUND OF THE INVENTION

1. Field of the Invention

The present invention relates to a novel lactic acid-forming microorganism which exhibits excellent probiotic properties. The present invention also relates to the uses of the novel microorganism as a probiotic in food, beverage, animal feed and/or dietary supplement compositions, and as a medicament in controlling the colonization of undesirable intestinal microorganisms in the alimentary tract of a mammal.

2. Description of the Related Art

The oral administration of large numbers of Lactobacillus rhamnosus, such as L. (casei subsp.) rhamnosus GG (ATCC 53103), to a mammal has been found helpful to maintain or even enhance the healthy state of the mammal. It is believed that L. rhamnosus, when ingested, would colonize transiently on the intestinal mucosa, which results in inhibition of the growth of pathogenic bacteria and viruses (such as rotavirus), stabilization of gut permeability, and suppression of allergic reactions in food hypersensitivity. The bacterium is particularly effective in alleviating the symptoms of gastroenteric disorders, such as diarrhea, by eliciting nonspecific humoral immune response in hosts.

L. rhamnosus, reported in 1989 as a new species derived from L. casei, shares similar phenotypes with two other members of the Lactobacillus genus, i.e., L. casei and L. paracasei. The three species can be further distinguished in terms of the differences in the genes encoding ribosomal RNAs. Approaches have been conducted based on this finding. For example, Rodtong et al. recognized the species-uniqueness of 16S rDNA and developed a ribotyping process to differentiate Lactobacillus strains (Rodtong, S. and Tannock, G. W. (1993) Applied and Environmental Microbiology 59: 3480-3484). Taking advantage of the convenience and effectiveness of polymerase chain reaction (PCR), Ward et al. and Alander et al., on the other hand, used different sets of primers to identify L. rhamnosus based on the sequence polymorphism of 16S rDNA (Ward, L. J. H., and Timmins, M. J. (1999) Letters in Applied Microbiology 29: 90-92; and Alander, M. et al., Applied and Environmental Microbiology 65: 351-354).

According to the present invention, the inventor has identified a novel strain of L. rhamnosus (hereinafter referred to as strain Tcell-1) which is phylogenetically distinct from the published strains in the species and exhibits excellent probiotic properties.

### SUMMARY OF THE INVENTION

It is a primary object of the present invention to provide a novel strain of L. rhamnosus. In the experiments performed in the invention, the inventor has characterized the phylogenetic distinction of the bacterial strain and demonstrated the desired probiotic properties thereof.

Another object of the present invention is to provide a composition containing the bacterium strain according to the invention and a suitable excipient for the manufacture of foodstuffs, such as beverages, food, animal feed, and dietary supplements.

Still another object of the present invention is to provide a pharmaceutical composition comprising the bacterium strain according to the invention, as well as to provide a method for the treatment or prophylaxis of gastroenteric disorders in a subject by administering such a composition to the subject.

### BRIEF DESCRIPTION OF THE DRAWINGS

These and other features and advantages of this invention will become apparent from the following detailed description of this invention, with reference to the accompanying drawings, in which:

FIGS. 1A-C demonstrate the enteroscopic sampling from the upper jejunum and rectum tissues of a volunteer;

FIG. 2 is a fermentation profile of the bacterial strain according to the present invention;

FIG. 3A is a restriction map of the chromosomal DNA from the bacterial strain according to this invention;

FIG. 3B shows the result of Southern analysis of FIG. 3A using E. coli MRE600 16S+23S rDNA as the probe;

FIG. 4 shows the result of PCR analysis using the primers designed by Ward & Timmins, in which the DNA extracted from the bacterial strain according to the invention (lane 2) and water (lane 3; as a negative control) was subjected to PCR;

FIG. 5 shows the result of PCR analysis using the primers designed by Alander et al., in which two sets of the species-specific primers, rham-rham2 (lanes 2-3) and rham-casei (lanes 4-5) were used in the PCR; and

### DETAILED DESCRIPTION OF THE INVENTION

In accordance with the present invention, a strain of L. rhamnosus was isolated from the intestinal specimens donated by domestic volunteers. In a preliminary process, the microorganisms from the specimens were screened by a series of selective media, among which MRS agar medium and Rogosa SL agar medium exclusively allow the proliferation of Lactobacillus. The bacteria selected according to the above procedure were subjected to a four-step screening strategy for identifying L. rhamnosus:
Step 1: fermentation patterning using an API 50CHL kit (BioM'erieux, Lyon, France);
Step 2: ribotyping according to the method described in Rodtong et al. (supra), in which the total DNAs extracted from the microorganisms were treated with restriction enzymes EcoRI, BclI, BglII or HindIII and detected by the rDNA probe of Escherichia coli subsequent to Southern blotting, so that the restriction fragment fingerprints of the suspected microorganisms can be obtained and compared with those derived from the L. rhamnosus DNA;
Step 3: PCR analysis according to the method described in Ward et al. (supra), in which a universal primer Y₂ (5′-CCCAC TGCTG CCTCC CGTAG GAGT-3′) and a species-specific primer rham (5′-TGCAT CTTGA TTTAA TTTTG-3′) were used in the reaction such that a major product of 290 bp will be produced when the chromosomal DNA of L. rhamnosus appears in the reaction mixture; and
Step 4: PCR analysis according to the method described in Alander et al. (supra), in which a pair of species-specific primers, rham (as indicated in Step 3) and rham2 (5′-CCGTC AATTC CTTTG AGTTT-3′), will amplify a specific product of 863 bp in the presence of L. rhamnosus DNA.

Accordingly, the bacterial strain according to this invention was identified to belong to the species L. rhamnosus. However, sequencing of the 863 bp product obtained in the Step 4 leads to a surprising finding that the bacterial strain according to this invention is phylogenetically distinct from all the published strains in the species L. rhamnosus.

The bacterial strain thus identified was designated as “Lactobacillus rhamnosus Tcell-1” and was deposited in the Culture Collection and Research Center (CCRC) of the Food Industry Research and Development Institute (FIRDI), Hsinchu, Taiwan, R.O.C. under the accession number CCRC 910145 (on Apr. 14, 2000). The bacterium was also deposited at the American Type Culture Center (ATCC) with accession number PTA-2406 on Aug. 22, 2000 under the terms of the Budapest Treaty on the International Recognition of the Deposit of Microorganism for the Purpose of Patent Procedure.

Further studies concerning the probiotic properties of the strain Tcell-1 were also conducted. The results reveal that the bacterial strain according to the present invention can survive and grow well even in the stringent environment that an ingested bacterium would encounter in the gastrointestinal tracts, including extremely acidic pH and a high level of bile. The ability of L. rhamnosus Tcell-1 to resist certain antibiotics is apparently beneficial to administration of the bacterium to a subject who is required to take antibiotics. The superiority of the bacterial strain is further reinforced by its capability of inhibiting the growth of various enterobacteria.

In view of the advantageous properties mentioned above, the bacterial strain according to the present invention is suitable for acting as a probiotic. According to the present invention, the strain Tcell-1 can be formulated into a broad variety of edible materials, including beverages, such as fluid milk, fermented milk, yogurts, fruit juices and sports drinks; food, such as ice cream, cheese and snacks; animal feed; dietary supplements; and infant formulas. Apparently, it is appreciable to those skilled in the art that the bacterial strain of this invention can be formulated in any suitable form by conventional methods for human or non-human animal's uptake. More preferably, the bacterial strain of this invention is formulated into the edible material in combination with other probiotic organisms, such as L. acidophilus, L. brevis, L. casei, L. plantarum, L. salivarius, L. bifidus, L. bulgaricus, L. causasicus, Streptococcus lactis and other strains of L. rhamnosus, or a combination thereof. In addition, L. rhamnosus Tcell-1 is preferably formulated together with lactosucrose, chitin, chitosan, manitol, yogurt powder or a combination thereof.

L. rhamnosus Tcell-1 can also be used alone or with other active ingredients as a medicament in controlling the colonization of undesirable intestinal microorganisms in the alimentary tract of a mammal, to alleviate the conditions caused thereby. The composition can be formulated in solution, emulsion, powder, tablet, capsule or other adequate forms for oral administration.

The following examples are given for the purpose of illustration only and are not intended to limit the scope of the invention.

### THE PREFERRED EMBODIMENTS OF THE INVENTION

### Example 1

### Isolation of L. rhamnosus Tcell-1

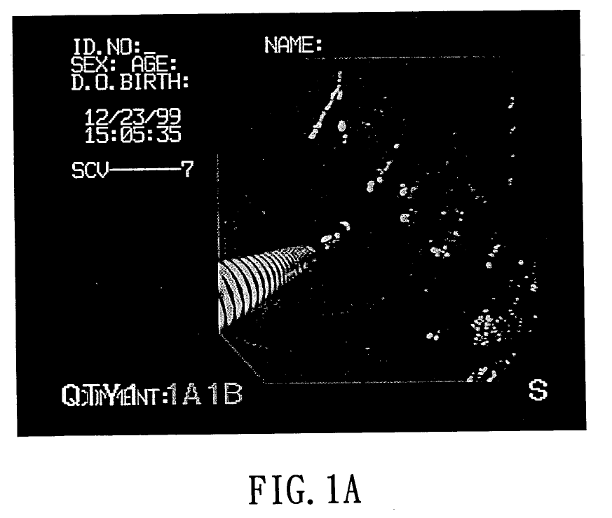

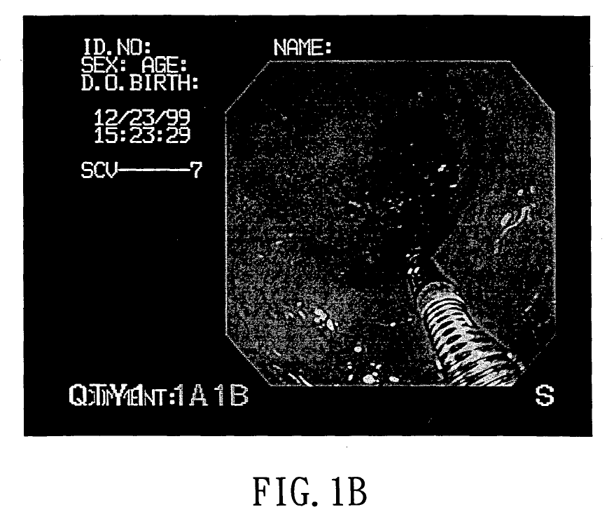

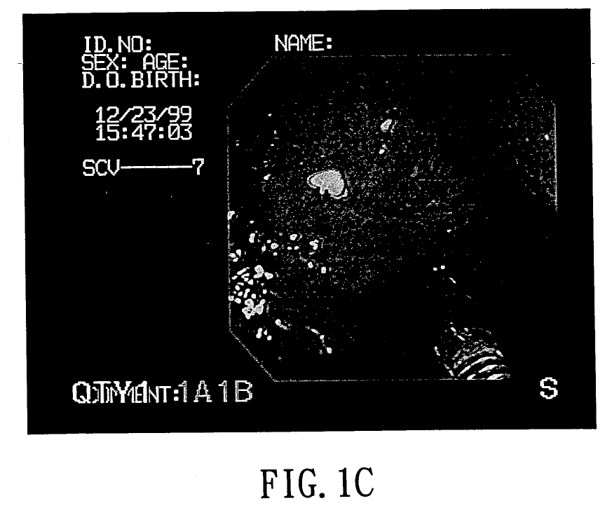

Six healthy adults, aged from 25-45 and having no addiction to alcohol or smoking or chronic use of a drug, participated voluntarily in this experiment. None of them are vegetarians nor have abnormal dietary habit. The voluntary donors were subjected to fasting for 12 hours before enteroscopic sampling. Three biopsy specimens, each about 2 mm² in size, were picked up from different sites in the upper jejunum and rectum of each donor (FIGS. 1A-C). The tissue specimens were then washed with physical saline (0.9% NaCl in distilled water) and stored in an ice-cold storage solution (0.9% NaCl, 0.1% Peptone, 0.1% Tween-80 and 0.02% Cysteine) for further analysis. The specimens were treated in an ultrasonic bath for 5 minutes and agitated vigorously for an additional 2 minutes. The obtained suspensions were undiluted or diluted in five- or ten-fold, and aliquots of the preparations were spread on the following solid media to obtain the profiles of enterobacteria contained therein (see also Johansson et al., Applied and Environmental Microbiology 59(1): 15-20).

1. Brain heart infusion agar (purchased from Difco) which is an enriched medium for aerobically or anaerobically culturing the entire population of enterobacteria at 37° C. for 3 days;

2. MRS agar (Difco) for anaerobically culturing Lactobacillus at 37° C. for 5 days;

3. Phenol ethanol agar (Difco) for aerobically or anaerobically culturing the Gram(+) bacteria at 37° C. for 3 days;

4. Azide blood agar (purchased from Oxoid) for aerobically culturing Streptococcus at 37° C. for 2 days;

5. Slanetz-Bartley agar (Oxoid) for anaerobically culturing Enterococcus at 37° C. for 2 days;

6. Violet red bile glucose agar (Oxoid) for culturing Enterobacteriaceae at 37° C. for 2 days;

7. Rogosa SL agar (Difco) for anaerobically culturing Lactobacillus at 37° C. for 5 days; and

8. Reinforced clostridial agar (Difco) for anaerobically culturing L. bifidus at 37° C. for 5 days.

The results are shown in Table 1.

TABLE 1
In the upper jejunum specimens:

| Name | Medium^{†} | | | | | | | | | | | | | | |
|---|---|---|---|---|---|---|---|---|---|---|---|---|---|---|---|
| of the | BHIA | | | MRS | | | PEA | | | ABA | | | SBA | | |
| Donor | 1x^{‡} | 5x | 10x | 1x | 5x | 10x | 1x | 5x | 10x | 1x | 5x | 10x | 1x | 5x | 10x |
| JF | 3 | 0 | 0 | 0 | 0 | 0 | 0 | 0 | 0 | 1 | 0 | 0 | 0 | 0 | 0 |
| JS | M^{§} | 30 | 1 | 0 | 0 | 0 | 0 | 0 | 0 | M | 26 | 0 | 52 | 6 | 3 |
| HK | 0 | 0 | 0 | 0 | 0 | 0 | 0 | 0 | 0 | 0 | 0 | 6 | 0 | 0 | 0 |
| V | 0 | 0 | 0 | 0 | 0 | 0 | 0 | 0 | 0 | 0 | 0 | 0 | 0 | 0 | 0 |
| B | 0 | 0 | 0 | 0 | 0 | 0 | 0 | 0 | 0 | 0 | 0 | 0 | 0 | 0 | 0 |
| RG | 0 | 0 | 0 | 0 | 0 | 0 | 0 | 0 | 0 | 0 | 0 | 0 | 0 | 0 | 0 |

| Name | Medium | | | | | | | | |
|---|---|---|---|---|---|---|---|---|---|
| of the | VRBGA | | | RA | | | RCA | | |
| Donor | 1x | 5x | 10x | 1x | 5x | 10x | 1x | 5x | 10x |
| JF | 0 | 0 | 0 | 0 | 0 | 0 | M | 0 | 0 |
| JS | 0 | 0 | 0 | 0 | 0 | 0 | M | 0 | 21 |
| HK | 0 | 0 | 0 | 0 | 0 | 0 | 0 | 0 | 0 |
| V | 0 | 0 | 0 | 0 | 0 | 0 | 0 | 0 | 0 |
| B | 0 | 0 | 0 | 0 | 0 | 0 | 0 | 0 | 0 |
| RG | 0 | 0 | 0 | 0 | 0 | 0 | 0 | 0 | 0 |

In the rectum specimens:

| Name | Medium | | | | | | | | | | | | | | |
|---|---|---|---|---|---|---|---|---|---|---|---|---|---|---|---|
| of the | BHIA | | | MRS | | | PEA | | | ABA | | | SBA | | |
| Donor | 1x | 5x | 10x | 1x | 5x | 10x | 1x | 5x | 10x | 1x | 5x | 10x | 1x | 5x | 10x |
| JF | M | 47 | 6 | M | 23 | 1 | 0 | 9 | 0 | M | 1 | 0 | 28 | 0 | 0 |
| JS | M | 15 | 1 | M | 8 | 0 | 0 | 12 | 0 | M | 0 | 1 | 0 | 1 | 0 |
| HK | M | 1 | 0 | 0 | 0 | 0 | 0 | 0 | 0 | 12 | 0 | 0 | 0 | 0 | 0 |
| V | M | 0 | 0 | 0 | 0 | 0 | 0 | 0 | 0 | 30 | 0 | 0 | 0 | 0 | 0 |
| B | M | 28 | 11 | M | 67 | 3 | 0 | 10 | 0 | M | 14 | 0 | M | 4 | 4 |
| RG | M | 0 | 0 | 0 | | 0 | 0 | 0 | 0 | 1 | 0 | 0 | 0 | 0 | 0 |

| Name | Medium | | | | | | | | |
|---|---|---|---|---|---|---|---|---|---|
| of the | VRBGA | | | RA | | | RCA | | |
| Donor | 1x | 5x | 10x | 1x | 5x | 10x | 1x | 5x | 10x |
| JF | M | M | M | 0 | 0 | 0 | M | M | M |
| JS | M | 15 | 0 | 0 | 0 | 0 | M | M | 0 |
| HK | 0 | 0 | 0 | 0 | 0 | 0 | M | 0 | 0 |
| V | 0 | 0 | 0 | 0 | 0 | 0 | M | 0 | 0 |
| B | M | M | M | M | 0 | 0 | M | M | M |
| RG | 0 | 0 | 0 | 0 | 0 | 0 | M | 0 | 0 |

^{†}The abbreviation BHIA represents brain heart agar; MRS represents MRS agar; PEA represents phenol ethanol agar; ABA represents azide blood agar; SBA represents Slanetz-Bartley agar; VRBGA represents violet red bile glucose agar; RA represents Rogosa SL agar; and RCA represents reinforced clostridial agar.
^{‡}1X, 5X and 10X are the dilution folds of the bacterial suspensions.
^{§}The letter M indicates that the number of bacterial colonies on the medium plate is higher than 100.

As shown in Table 1, the profiles of enterobacteria in the upper jejunum and rectum specimens are quite different.

### Example 2

### Isolation of Lactobacillus

From the MRS and Rogosa SL agar media in Example 1, 200 colonies were picked up randomly and transferred separately to fresh MRS agar media containing 1% CaCO₃. After incubation, the colonies surrounded by clear zones were picked up, and each of them was transferred to a basal MRS agar medium supplemented with 1% rhamnose and 0.05% chlorophenol red. Finally, the yellowish colonies, presumably constituted by Lactobacillus, were picked up and further transferred to fresh MRS broth and incubated anaerobically at 37° C. for 2 days for further analysis.

### Example 3

### Identification of L. rhamnosus Tcell-1 as a New Strain

(a) Fermentation Pattern

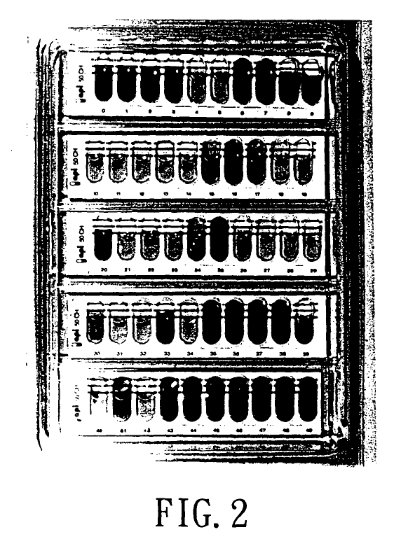

The Lactobacillus broth prepared in Example 2 was precipitated, washed with distilled water and resuspended in a defined amount of distilled water. The bacterial suspensions thus obtained were investigated using an API 50CHL kit according to the protocol provided by the manufacturer. Upon this procedure, a strain of L. rhamnosus was identified based on the fermentation pattern specific to the species (FIG. 2) and designated as L. rhamnosus Tcell-1.

Total DNA of the strain Tcell-1 was prepared from a 3 ml culture growing in the mid-log phase according to a conventional method described by Sambrook et al. (Sambrook, J. et al., Molecular Cloning, A Laboratory Manual, 2^{nd} ed., Cold Spring Harbor Laboratory Press, 1989), and resuspended in 50 μl of TE buffer (1 mM EDTA, 10 mM Tris-HCl, pH 8.0). The DNA solution thus obtained was used in the following analyses for further investigation of the strain Tcell-1.

(b) Ribotyping Analysis

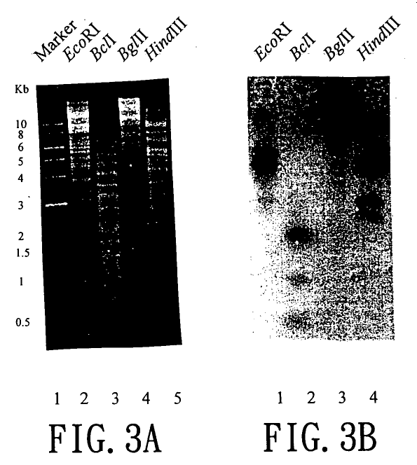

10 μl aliquots of the DNA were digested by restriction endonucleases, EcoRI, BclI, BglII and HindIII, respectively, for 3 hours. The digested products were loaded into the wells of a 0.8% agarose gel, and electrophoresis was carried out at 5 V/cm for 2 hours. The gel was then stained with ethidium bromide, and an image of the gel was obtained as shown in FIG. 3A. The DNAs on the gel were denatured and transferred to a nylon-based membrane (Hybond-N⁺, Amersham) as described by Sambrook et al. (supra). To prepare the probe for Southern analysis, 1 μl of E. coli MRE600 16S+23S rRNA (purchased from Boehringer Mannheim) was used as the template which was amplified via incorporation of [α-³²P] dCTP by AMV reverse transcriptase (Bethesda Research Laboratories) and random primers. Blots were hybridized at 68° C. for 16-24 hours in a hybridization solution containing 5×SSC, 1× Denhardt's solution, 1% SDS and 100 mg/ml of Harpin sperm DNA, washed properly to enhance the signal-to-noise ratio, and subjected to autoradiography. As shown in FIG. 3B, the ribotype of the strain Tcell-1 was in perfect agreement with the typical pattern of L. rhamnosus as described by Rodtong et al. (supra).

(c) PCR Analysis Using the Ward & Timmins' Primers

To an 1 ml eppendorf, 1 μl of Tcell-1 DNA harvested in Example 3(a), 1 μl of primer Y₂, 1 μl of primer rham, 0.5 μl of DynaZymeII (Finnzymes Oy) and each dNTP (dATP, dTTP, dCTP and dGTP) at 100 μM were added. The reaction mixture was added with distilled water to a final volume of 50 μl and further overlaid with mineral oil. The reaction mixture was placed in a GeneAmp® PCR System 2400 thermocycler (Perkin Elmer) and thermocycled under the following conditions:

| | |
|---|---|
| Initial condition: | 94° C. for 3 min. |
| | 45° C. for 45 sec. |
| | 72° C. for 1 min. |
| Thermocycling: | 94° C. for 45 sec. |
| | 45° C. for 45 sec. |
| | 72° C. for 1 min. |
| Number of thermocycles: | 30 |
| Chain extension: | 94° C. for 45 sec. |
| | 45° C. for 45 sec. |
| | 72° C. for 5 min. |

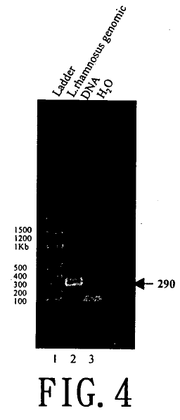

Following thermocycling, the amplified products were separated on a 0.2% agarose gel. The gel was stained with ethidium bromide, and a major amplicon of 290 bp was observed on the gel under a UV light source (FIG. 4)

(d) PCR Analysis Using the Alander's Primers

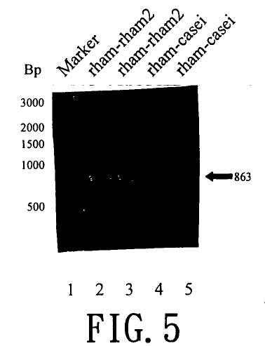

The PCR in Example 3 (c) was repeated except that the Ward & Timmins' primers were replaced with the rham and rham2 primers designed by Alander et al. (supra). The electrophoresis analysis on a 0.2% agarose gel revealed that the amplified products contain a major band of 863 bp (FIG. 5), which, as described above, was reported to be a critical indicator to identify L. rhamnosus.

The experiments conducted in Examples 3(a)-(d) conclude that the biochemical and genetic traits of the strain Tcell-1 matched with those considered belonging to species L. rhamnosus.

(e) Differentiation of Strain Tcell-1 from other L. rhamnosus Strains

Using TOPO TA cloning™ kit (Invitrogen), the 863 bp product obtained in Example 3(d) was cloned into a pCR-TOPO™ vector according to the protocol provided by the manufacturer. The resultant plasmid was then introduced into TOP10 One Shot™ electrocompetent cells (Invitrogen) by electroporation. Following proliferation of the transformants in a selective medium, the plasmid was harvested and the 863 bp insert was sequenced.

The sequence was used as a query sequence and searched against a nucleotide sequence database in the GenBank (http://www.ncbi.nlm.nih.gov:80/entrez/query.fcgi?CMD=search&DB=nucleotide). The sequence alignment shown in Table 2 suggests that the strain Tcell-1 is phylogenetically distinct from all the six L. rhamnosus strains available in the GenBank based on the 16S rDNA sequences. The Tcell-1 DNA sequence shown in Table 2, which is 776 bp in size, was designated as SEQ ID No.1.

TABLE 2

symbol comparison table: genetiq.dat; gap penalty: 4

| | |
|---|---|
| | 1 10 20 30 40 50 |
| TCELL1 | TATACACTGGTACCTCCCTAAGTGGGATACATTGAAACAATCTATCCGCATAATCAAGA |
| | *** *** * * * * * * * * |
| AF21761 | TTGTACACACCGCCC.GTCACACCATGAGAGTTTGTAACA...CCCGAAGCCGGTG |
| | *************** ************************ ************* |
| E08782 | CTTGTACACACCGCCC.GTCACACCATGAGAGTTTGTAACA...CCCGAAGCCGGTG |
| | * * * * * * * * ** *** * **** * |
| AF18273 | CCTTTCTAAGGAAACAGACTGAAAGTCTGACGGAAACCTGCACA...CACGAAACTTTGT |
| | * * *** **** ** |
| A61362 | CTAAGGAAACAGACTGAAAGTCTGACG................ |
| | *************************** |
| U32966 | CTAAGGAAACAGACTGAAAGTCTGACG................ |
| | ************************* |
| AF12120 | AAGGAAACAGACTGAAAGTCTGACG................ |
| | 1 10 20 |
| consens | A C CC A GA ACAGAC GAAA TCT AC C C A |
| | 1 10 20 30 40 50 60 |
| | 70 80 90 100 110 |
| TCELL1 | CCGCATGTCTTGCTAAGATGCGTAACTATCGCTTTGGATGACCCCGCGTATAGCTAGTTG |
| | ** * * * * * * * ** * * |
| AF21761 | GCGTAA......CCCTTTTAGGGAGCGA.............GCCGTCTAAGGTGGGACAA |
| | ****** ** *** ** * * * ** ** |
| E08782 | GCGTAA......CCTTTTAGGGAGCGAG.............CCGTCTAAGGTGGGACAAA |
| | ** * * |
| AF18273 | TTAGTTTTGAGGGGATTACCCTCAAGCACCC.........TAGCGGGTGCGACTTTGTTC |
| A61362 | ............................................................. |
| U32966 | ............................................................. |
| AF12120 | ............................................................. |
| consens | |
| | 70 80 90 100 110 120 |
| | 130 140 150 160 170 |
| TCELL1 | TAAGTAACGCTCACCAAGCAATGATGCTAGCCAACTAAGTTGATCGCCACATTGGACTAA |
| | * * * * * * ** * * * * * ** * * ** |
| AF21761 | ATGATTAGGGTGAAGTCGTAACAAGGTAGCCGTAGGAGAACCTGCGGCTGGATCACCTCC |
| | * ** * ***************************************** |
| E08782 | TGATTAGGGTGAAGTCGT.AACAAGGTAGCCGTAGGAGAACCTGCGGCTGGATCACCTCC |
| | * * ** * * * * ** * * * |
| AF18273 | TTTGAAAACTGGATATCATTGTTGTAAATGTTTTAAATTGCCGAGAACACAGGCTATTTG |
| A61362 | ............................................................ |
| U32966 | ............................................................ |
| AF12120 | ............................................................ |
| consens | A A C T |
| | 130 140 150 160 170 180 |
| | 190 200 210 220 230 |
| TCELL1 | ACACGGCC.CAAACTCTACGGAGGCAGCAGTAGGAATCTTCCACAATGGACGCAAGTCTG |
| | * **** ** ** * ** * * |
| AF21761 | TTTCTAAG.GAAACAG.ACTGAAAGTCTGA...........CGGAAACCTGCACACACGA |
| | ******** ******* ************* ******************* |
| E08782 | TTTCTAAG.GAAACAG.ACTGAAAGTCTGA...........CGGAAACCTGCACACACGA |
| | * * ** * * * * ** * * * ** ** * * |
| AF18273 | TATGAGTTTCTAATAATAGAAATTCGCAT............CGCATAACCGCTGACGCAA |
| | * * * ** ** * * |
| A61362 | ...........................................GAAACCTGCACACACGA |
| | ***************** |
| U32966 | ...........................................GAAACCTGCACACACGA |
| | ***************** |
| AF12120 | ...........................................GAAACCTGCACACACGA |
| | 30 40 |
| consens | AA A A C GAAACCTGCACACACGA |
| | 190 200 210 220 230 240 |
| | 250 260 270 280 290 |
| TCELL1 | ATGGAGCAACGCCGCGTGACTGAAGAAGGCTTTCGGGGCGTAAAACTCTGTTGTTGGAGA |
| | * ** * * * ** |
| AF21761 | AACTTTGTTTAGTTTTGAGGGGATTACCCTCAAGCACCCTAGCGGGTG.......CGACT |
| | ************************ **************** ****** ***** |
| E08782 | AACTTTGTTTAGTTTTGAGGGGATCACCCTCAAGCACCCTAACGGGTG.......CGACT |
| | * * * * * * * ** *** |
| AF18273 | GTCAGTACAGGTTAAGTTACAAAGGGCGCACGGTGGATGCCTTGGCACTAGGAGCCGATG |
| | * * * * * * * ** |
| A61362 | AACTTTGTTTAGTTTTGAGGGGATTACCCTCAAGCACCCTAGCGGGTG............ |
| | ************************************************ |
| U32966 | AACTTTGTTTAGTTTTGAGGGGATTACCCTCAAGCACCCTAGCGGGTG............ |
| | ************************************************ |
| AF12120 | AACTTTGTTTAGTTTTGAGGGGATTACCCTCAAGCACCCTAGCGGGTG............ |
| | 50 60 70 80 90 |
| consens | AACTTTGTTTAGTTTTGAGGGGATTACCCTCAAGCACCCTAGCGGGTG GA |
| | 250 260 270 280 290 300 |
| | 310 320 330 340 350 |
| TCELL1 | AGAATGGTCGGCAGAGTAACTGTTGTCGGCGTGACGGTATCCAACCAGAAAGCCACGGCT |
| | ***** ** * * * * |
| AF21761 | TTGT.....TCTTTGAAAACTGGATATCATTGTTGTAAATGTTTTAAATTGCCGAGAACA |
| | **** ************************ *** ********************* |
| E08782 | TTGT.....TCTTTGAAAACTGGATATCATTGTATTAATTGTTTTAAATTGCCGAGAACA |
| | * ** * * ** * ** ** * * ** |
| AF18273 | AAGGACGGAACTAATACCGATATGCTTCGGGGAGCTATAAGTAAGCTTTGATCCGGAGAT |
| | ** * * * |
| A61362 | ...............................CGACTTTGTTCTTTGAAAACTGGATATCA |
| | ***************************** |
| U32966 | ...............................CGACTTTGTTCTTTGAAAACTGGATATCA |
| | ***************************** |
| AF12120 | ...............................CGACTTTGTTCTTTGAAAACTGGATATCA |
| | 100 110 |
| consens | T GACT T T CTTT AAAA TCGA A CA |
| | 310 320 330 340 350 360 |
| | 370 380 390 400 410 |
| TCELL1 | AACTCAGTGCCAGCAGCCGCGGTAATACGTAGGTGGCAAGCGTTATCCGGATTTATTGGG |
| | * * * * |
| AF21761 | CAGCGTATTTGTATGAGTTTCTAATAA..............................TA |
| | ***** ** ** ** * |
| E08782 | CAGCG.TATTTGTATGAGTTTCTGAAA..............................AA |
| | ** * * * * |
| AF18273 | TTCCGAATGGGGGAACCCAGTACACATCAGTGTATT.....................GC |
| | ** |
| A61362 | TTGTTGTAA.................................................AT |
| | ********* ** |
| U32966 | TTGTTGTAA.................................................AT |
| | ********* ** |
| AF12120 | TTGTTGTAA.................................................AT |
| | 130 |
| consens | TTGT TA A |
| | 370 380 390 400 410 420 |
| | 430 440 450 460 470 |
| TCELL1 | CGTAAAGCGAGCGCAGGCGGTTTTTTAACTCTGATGTGAAAGCCCTCGGCTTAACCGAGG |
| | * * **** ** * ** * * |
| AF21761 | GAAATTCGCATCGCA.......................TAACCGCTGACGCAAGTC.... |
| | *************** ****************** |
| E08782 | GAAATTCGCATCGCA.......................TAACCGCTGACGCAAGTC.... |
| | * ** ** * * |
| AF18273 | CTGCAAGTGAATACA.......................TAGCTTGTTGGCGGCAGACGCG |
| | * * * |
| A61362 | GTTTTAAATTGCCGA................................................ |
| | *************** |
| U32966 | GTTTTAAATTGCCGA................................................ |
| | *************** |
| AF12120 | GTTTTAAATTGCCGA................................................ |
| | 140 |
| consens | GTT TA AGC CA A T |
| | 430 440 450 460 470 480 |
| | 490 500 510 520 530 |
| TCELL1 | AAGTGCATCGGAAACTGGGAAACTTGAGTACAGAAGAGGACAGTGGAACTCCATGTGTAG |
| | * ** * * * ** * * * |
| AF21761 | AGTACCAGGTAAGTTACAAAGGGCGCACGGTGGATGCCTTGGCACTAGGAGC.......C |
| | ***** * ******************************************* * |
| E08782 | AGTACAGGTTAAGTTACAAAGGGCGCACGGTGGATGCCTTGGCACTAGGAGC.......C |
| | * * * * * ** **** * * * * * * * |
| AF18273 | GGGAACTGAAACATCTCAGTACCCGCAGGAAGAGAAAGAAAACTCGATTCCCATAGTAGC |
| | **** * * ** * * ** * * |
| A61362 | ..GAACACAGCGTATTTGTATGAGTTTCTAATAATAGAAATTCGCATC............ |
| | ********************************************** |
| U32966 | ..GAACACAGCGTATTTGTATGAGTTTCTAATAATAGAAATTCGCATC............ |
| | ********************************************** |
| AF12120 | ..GAACACAGCGTATTTGTATGAGTTTCTAATAATAGAAATTCGCATC............ |
| | 150 160 170 180 190 |
| consens | GAACA AG ATT G A G T ACTAA AATA AA C C A C |
| | 490 500 510 520 530 540 |
| | 550 560 570 580 590 |
| TCELL1 | CGGTGAAATGCGTAGATATATGGAAGAACACCAGTGGCGAAGGCGGCTGTCTGGTCTGTA |
| | * * * *** ** * * * * * * |
| AF21761 | GATGAAGGACGGAACTAATACCGATATGCTTCGGGGAGCTATA................A |
| | ******************************************* * |
| E08782 | GATGAAGGACGGAACTAATACCGATATGCTTCGGGGAGCTATA................A |
| | * ** ** * * ** * * * *** |
| AF18273 | GGCGAGCGAAGTGGGAAGAGCCCAAACCGAGAAGCTTGCTTCTCGGGGTTGTAGGACTGG |
| | * * * * * ** * * * |
| A61362 | .............GCATAACCGCTGACGCAAGTCAGTACAGG |
| | ***************************** |
| U32966 | .............GCATAACCGCTGACGCAAGTCAGTACAGG |
| | ***************************** |
| AF12120 | .............GCATAACCGCTGACGCAAGTCAGTACA |
| | 200 210 218 |
| consens | GCATAA CGCA ACGCA G GT CA |
| | 550 560 570 580 590 600 |
| | 610 620 630 640 650 |
| TCELL1 | ACTGACGCTGAGGCTCGAAAGCATGGGTAGCGAACAGGATTAGATACCCTGGTAGTCCAT |
| | * *** * * * * **** * ** |
| AF21761 | GTAAGCTTTGATCCGGAGATT...........TCCGAATGGGGGAACCCAGTA....CAC |
| | ********************* ********************* *** |
| E08782 | GTAAGCTTTGATCCGGAGATT...........TCCGAATGGGGGAACCCAGTA....CAC |
| | * ** ** * * * * * * * * * * |
| AF18273 | ACATTGGAGTTACCAAAGTTCG..........ACGTAGTCGAAGTCAGCTGGAAAGCTGC |
| A61362 | |
| U32966 | |
| AF12120 | |
| consens | C C G |
| | 610 620 630 640 650 660 |
| | 670 680 690 700 710 |
| TCELL1 | GCCGTAAACGATGAATGCTAGGTGTTGGAGGGTTTCCGCCCTTCAGTGCCGCACTAACGC |
| | * * *** * ** * * * * **** |
| AF21761 | ATCAGTG.....TATTGCCTGCAAGTGAATACATAGCTTGT......TGGCGGCAGACGC |
| | ******* * * ** * * * * * * |
| E08782 | ATCAGTGTGTTGCTTGTCAGTGAATACATAGCTGGCCGGCG......GCCAGACGCGGGG |
| | ** * * ** * ** * * * * * |
| AF18273 | GCCATAGAAGGTGAAAGCCCTGTAAACGAAACGGCGGACTC....TCCGTCCAGGATCCT |
| A61362 | |
| U32966 | |
| AF12120 | |
| consens | C C |
| | 670 680 690 700 710 720 |
| | 730 740 750 760 770 776 |
| TCELL1 | ATTAAGCATTCCGCCTGGGGAGTACGACCGCAAGGTTGAAACTCAAAGGAATTGACGG |
| | * * ** * ** |
| AF21761 | GGGGAACT......................GAAACATCTAAG |
| | * * * ** |
| E08782 | AACTGAAA......................CATCTAAGTACCCGGA |
| | * * * * * * |
| AF18273 | GAGTACGGCGGAACACGTGAAATTCCGTCGGAATCCGGGAGGACCATCT |
| A61362 | |
| U32966 | |
| AF12120 | |
| consens | A A |
| | 730 740 750 760 770 778 |

### Example 4

### Characterization of L. rhamnosus Tcell-1

(a) Tolerance of Acid

MRS liquid media were prepared at pH 2, 3, 4, 5 and 6, respectively, and supplemented with 0.3% bile salt. To 1.5 ml of each medium, 10⁶ Tcell-1 cells were inoculated and incubated anaerobically at 37° C. Samples were collected at 0 and 4 hours after the inoculation, and the populations of the microorganisms in each culture were assessed with reference to the optical density at 620 nm. The results are shown in Table 3.

TABLE 3

| Incubation | | OD₆₂₀ | | | | |
|---|---|---|---|---|---|---|
| | time | pH2 | pH3 | pH4 | pH5 | pH6 |
| | 0 hour | 0.01 | 0.018 | 0.010 | 0.014 | 0.012 |
| | 4 hour | 0.01 | 0.018 | 0.016 | 0.025 | 0.030 |

As shown in Table 3, the growth rate of L. rhamnosus Tcell-1 remarkably reduced as the cultures were acidified to a pH at which the ingested substances would encounter in an animal stomach, i.e., a pH of below 3. Surprisingly, the cells incubated in such an acidic environment for 4 hours can still restore their normal growth if transferred to a fresh MRS medium at pH 6.0 (data not shown). The data indicate that L. rhamnosus Tcell-1 can tolerate the attack of gastric acid.

(b) Tolerance of Bile Salt

Example 4(b) was repeated except that the MRS liquid media contained bile salts at concentrations of 0.1, 0.2, 0.3 and 0.4%, respectively, while the pH of the media was constantly set at 2.5. The results are shown in Table 4.

TABLE 4

| | |
|---|---|
| Incubation | OD₆₂₀ |

| time | 0.1% | 0.2% | 0.3% | 0.4% |
|---|---|---|---|---|
| 0 hour | 0.011 | 0.010 | 0.013 | 0.018 |
| 4 hour | 0.028 | 0.025 | 0.023 | 0.032 |

From Table 4, it is demonstrated that the growth of L. rhamnosus Tcell-1 was sustained at a high level of bile.

(c) Resistance to Antibiotics

2-3 ml aliquots of a bacterial suspension from an overnight culture of L. rhamnosus Tcell-1 were spread on MRS agar media added with 10 μg/ml of kanamycin, vancomycin, chloramphenicol or ampicillin. After incubation, L. rhamnosus Tcell-1, while its growth was attenuated in the media containing chloramphenicol or ampicillin, was found to be tolerant of kanamycin and vancomycin.

(d) Inhibition of the Colonization of Other Bacteria

2-3 ml aliquots of a bacterial suspension from an overnight culture of L. rhamnosus Tcell-1 were spread on MRS agar media. Each of the plates was incubated at 30° C. for 22 hours, on which 7 ml of soft agar mixed with 100 μl suspension from one of the nine enterobacterial strains listed in Table 5 (purchased from the FIRDI) was poured.

TABLE 5

| Bacterium | Medium |
|---|---|
| Enterobacter aerogenes | DIFCO 0001 |
| Clostridium perfringens | brain heart infusion |
| | (anaerobically cultured) |
| Klebsiella pneumoniae | DIFCO 0001 |
| Yersinia enterocolitica | brain heart infusion |
| Listeria monocytogenes | brain heart infusion |
| Streptococcus mutans | brain heart infusion |
| Citrobacter freundii | DIFCO 0001 |
| Shigella dysenteriae | DIFCO 0001 |
| Yersinia ruckeri | DIFCO 0001 |

The obtained cultures were incubated for an additional 48 hours at 37° C. and observed with bare eyes. Based on the presence of inhibition rings around the colonies of L. rhamnosus Tcell-1, the inventor found that the strain Tcell-1 can significantly suppress the growth of E. aerogenes, C. perfringens, L. monocytogenes, S. mutans and C. freundii. The results strongly suggest that L. rhamnosus Tcell-1 exhibits promising probiotic properties for controlling or inhibiting the colonization of the undesired bacteria in the bowel.

### Example 5

### Probiotic Formulations Containing L. rhamnosus Tcell-1

L. rhamnosus Tcell-1 can be utilized in various forms of foodstuffs, two examples of which are described as follows:

Formula 1:

Ten Strains of Lactic Acid-Forming Bacteria:

L. acidophilus, L. brevis, L. casei, L. plantarum, L. salivarius, L. bifidus, L. bulgaricus, L. causasicus, Streptococcus lactis and L. rhamnosus Tcell-1;
   Excipients:
lactosucrose oligo, manitol, chitin & chitosan, yogurt powder;
   Natural Condensates:
alfalfa, barley and wheat grass juice powder, pure soya lecithin, carrot juice powder, phosphatidyl choline, Hawaiian Spirulina pacifica, apple pectin powder, phosphatidyl inositol CGF chlorella, non-dairy probiotic culture: rhamnosus, acidophilus in a base of FOS, peace river bee pollen powder, stevia, freeze-dried mango, black currant, dandelion root extract 4:1, beetroot extract, Siberian ginseng extract 0.4%, pacific kelp 4:1 extract, artichoke 4:1 extract 2%, soya extract, bilberry extract 5:1, pineapple extract, cranberry juice extract 18:1, rosehip extract 4:1, lycopene, Milk Thistle Phytosome™, Ginkgo Biloba Phytosome™ and Grape Seed Phytosome™;
   Other Ingredients:
Vitamin C, calcium, magnesium, zinc.
   Formula 2:
   Nine Strains of Lactic Acid-Forming Bacteria:
L. acidophilus, L. brevis, L. casei, L. plantarum, L. salivarius, L. bifidus, L. bulgaricus, L. causasicus and L. rhamnosus Tcell-1;
   Other Ingredients:
calcium lactate, lactosucrose oligo, lactose, dextrose, powered milk, vegetable oil and small amounts of an emulsifier and natural seasonings

The formulation is coated on peanut and raisin granules to make up a probiotic healthy dessert.

With this invention thus explained, it is apparent that numerous modifications and variations can be made without departing from the scope and spirit of this invention. It is therefore intended that this invention be limited only as indicated by the appended claims.

1. A bacterial isolate of Lactobacillus rhamnosus which is deposited in Food Industry Research and Development Institute (FIRDI) with an accession number CCRC 910145 and in American Type Culture Center (ATCC) with an accession number PTA-2406.

2. A composition containing the bacterial isolate of claim 1, and an acceptable excipient.

3. The composition of claim 2, which is in a form selected from the group consisting of beverages, food, animal feed, and dietary supplements.

4. The composition of claim 2, wherein the excipient is lactosucrose, chitin, chitosan, manitol, yogurt powder or a combination thereof.

5. The composition of claim 2, wherein the bacterial isolate is formulated into the excipient together with a probiotic organism selected from the group consisting of L. acidophilus, L. brevis, L. casei, L. plantarum, L. salivarius, L. bifidus, L. bulgaricus, L. causasicus, Streptococcus lactis and other strains of L. rhamnosus, or a combination thereof.

6. A pharmaceutical composition comprising a probiotic amount of the bacterial isolate of claim 1.

7. The pharmaceutical composition of claim 6 for use in the treatment or prophylaxis of gastroenteric disorders.

8. A method for the treatment or prophylaxis of gastroenteric disorders in a subject in need of such treatment or prophylaxis comprising administering to the subject a composition containing a probiotic amount of the bacterial isolate of claim 1.
